(19) United States
(12) Patent Application Publication
(10) Pub. No.: US 2013/0236430 A1
(43) Pub. Date: Sep. 12, 2013

(54) PROLIFERATION, SEPARATION AND TRANSPLANTATION OF INHIBITORY NEURON PROGENITORS AND PROLIFERATION PROMOTING SUBSTANCES FOR THE PROGENITORS

(71) Applicants: Nobuaki Tamamaki, Kumamoto (JP); Shengx Wu, Kumamoto (JP); Shogo Ninomiya, Kumamoto (JP)

(72) Inventors: Nobuaki Tamamaki, Kumamoto (JP); Shengx Wu, Kumamoto (JP); Shogo Ninomiya, Kumamoto (JP)

(21) Appl. No.: 13/825,409

(22) PCT Filed: Sep. 22, 2011
(86) PCT No.: PCT/JP2011/072490

§ 371 Date: May 28, 2013

(30) Foreign Application Priority Data
Sep. 22, 2010 (JP) 2010-212133

(51) Int. Cl. A61K 38/22

(52) U.S. Cl. CPC A61K 38/22
USPC 424/93.7; 530/300; 435/406; 514/9.8; 435/381; 435/377; 435/7.1; 506/9

(57) ABSTRACT

Those provided here are an Activin A-containing solution for making inhibitory neuron progenitors proliferate, a method for culturing the inhibitory neuron progenitors using this solution, a method for separating the inhibitory neuron progenitors and the inhibitory neurons using an Activin A receptor Acvr1 expression as an index, a method for transplanting thus separated cells to the brain surface, and a method for screening a factor for making the inhibitory neuron progenitors proliferate.

### TECHNICAL FIELD

The invention of the present application relates to a solution for making inhibitory neuron progenitors proliferate in vivo and in vitro, a method for separating inhibitory neuron progenitors and inhibitory neurons from a cell population, a method for transplanting the separated cells, and a method for screening a substance that selectively proliferate inhibitory neuron progenitors.

### BACKGROUND ART

Most of the neurons in the central nervous system of a higher vertebrate cannot be regenerated after damaged, and their dysfunctions become chronic with no successful healing. For the purpose of healing such damage, it is attempted to differentiate embryonic stem (ES) cells or induced pluripotent stem (iPS) cells to neurons which are then transplanted (Non-Patent Documents 1 and 2). However, there are a very small number of neurons which have been differentiated, if possible, from ES cells or iPS cells, and that obtained actually is just a mixture of ES cell or iPS cell with various neural cells. While it is important for a transplantation to provide only a certain type of neuron progenitors and neurons, a difficulty is experienced also in the case of inhibitory neurons, and it has not been successful so far to obtain highly pure inhibitory neuron progenitors and developing inhibitory neurons.

The neurons in central nerve system include excitatory neurons and inhibitory neurons. The both are distributed at various ratios depending on the regions of the central nervous system and work for information processing. In cerebral cortex, the inhibitory neurons release gamma-aminobutyric acid (GABA) as a neurotransmitter while the excitatory neurons release glutamic acid. The inhibitory neurons compose the neocortex at a rate of about 20% of the neocortical neurons, and maintain an appropriate activity of the neocortical circuit, resulting in a smooth information processing. However, all neurons may sometimes begin to excite, resulting in an epileptic seizure accompanied by the loss of consciousness. While some causes of such an attack includes a febrile spasm caused by fever-induced neuron excitatory tendency due to a immature brain circuit in childhood, most are based on genetic backgrounds, and it is believed that many epileptic patients are susceptible to excitation because of a point mutation in a channel molecule involved in the excitation of neurons. Also when the molecular mechanism of cell migration is abnormal, the cerebral cortex grey matter is divided into two parts which lead to a loss of balance in the input-output relationships, resulting in a repetitive epilepsy-like attack. In any case, a short-circuit like abnormal condition in the neural circuit is suspected, and an excessive firing may lead to an influx of a large amount of calcium ion into a cell body, resulting in a cell death. Especially, the inhibitory neurons, which serve to prevent such the short circuit, is subjected to an excessive input, and undergoes an earlier death compared with the excitatory neurons. Such a short circuit is referred to as a refractory epileptic attack focus, and the inhibitory neurons in the focus are reduced drastically, thereby giving a repetitive epileptic attack source. In intractable epilepsy, no therapeutic medication is effective, and a radical treatment involving the excision of the focal region to suppress the epileptic attack onset is conducted, but the excision of a part of the brain results in a loss of the brain functions. If inhibitory neuron progenitors and developing inhibitory neurons could be transplanted and engrafted to such an epileptic attack focus, the epileptic attack suppression would become hopeful.

The inventors of this application have found that inhibitory neurons in a rodent cerebral cortex originate from basal ganglion primordium, and reported the finding on Nov. 1, 1997 (Non-Patent Documents 3). Independently, Anderson S. in United States reported the same phenomenon on Oct. 27, 1997 (Non-Patent Document 4). However, it was not confirmed that there is no origin other than the basal ganglion primordium. In addition, another group reported that 65% of the neocortical inhibitory neurons are produced in the human cerebral cortex and the rest are produced in the basal ganglion primordium (Non-Patent Documents 5). They speculated that 65% of the neocortical inhibitory neurons are produced as a result of the division of the inhibitory neuron progenitors in the ventricular zone or the subventricular zone and characterized by expression of Mash1. Although such findings are partly in consistent with the individual findings in the latest study of the inventors that investigates the origin of the rodent inhibitory neurons (Non-Patent Document 6), the interpretation of the origin is different from the present inventors. Based on the study in rodent by the present inventors, the cells migrating to cerebral cortex includes inhibitory neuron progenitors and developing neurons. The former supplies new inhibitory neurons in the cerebral cortex, thus the origin of all the inhibitory neurons in the mouse cerebral cortex should be the basal ganglion primordium. Also in the case of a human, the inhibitory neuron progenitors and inhibitory neurons of cerebral cortex are highly possible to be derived from the basal ganglion primordium.

Thus, it is increasingly becoming clear how the human inhibitory neuron progenitors and inhibitory neurons are derived, but it is still highly difficult to provide the inhibitory neuron progenitors and premature inhibitory neurons in an amount sufficient to accomplish a transplantation for the purpose of a treatment.

Activin A is well known as a substance involved in the formation of a dorsal-ventral axis during blastocyst stage and the formation of a kidney, and patent applications have been filed as to a molecule for forming a kidney (Patent Document 1), a inhibitory neuron protecting effect (Patent Document 2) and an adjustment of synaptic connection plasticity (Patent Document 3). However, these effects of Activin A are the events which are independent of each other, and the receptors to which Activin A binds to exert its effects on the respective cells are different. In addition, Activin A has not been known to make inhibitory neuron progenitors proliferate selectively at a high efficiency.

### Patent Document 1: JP-A 2008-505643

### Patent Document 2: JP-A 2002-524402

### Patent Document 3: JP-A 2003-130869

### Patent Document 4: JP-A 2004-229523

Non-Patent Document 1: Gaspard N, Bouschet T, Hourez R, Dimidschstein J, Naeije G, van den Ameele J, Espuny-Camacho I, Herpoel A, Passante L, Schiffmann S N, Gaillard A, Vanderhaeghen P. (2008) An intrinsic mechanism of corticogenesis from embryonic stem cells. Nature 455: 351-357.
Non-Patent Document 2: Takahashi K, Tanabe K, Ohnuki M, Narita M, Ichisaka T, Tomoda K, Yamanaka S. (2007) Induction of pluripotent stem cells from adult human fibroblasts by defined factors. Cell 131, 861-872.
Non-Patent Document 3: Tamamaki N, Fnjimori K, Takauji R. (1997) Origin and route of tangentially migrating neurons in the developing neocortical intermediate zone. J Neurosci 17:8313-8323.
Non-Patent Document 4: Anderson S A, Eisenstat D D, Shi L, Rubenstein J L R. (1997) Interneuron migration from the basal forebrain to the neocortex: dependence on Dlx genes. Science 278: 474-476.
Non-Patent Document 5: Letinic K, Zoncu R, Rakic P. (2002) Origin of GABAergic neurons in the human neocortex. Nature 417: 645-649.
Non-Patent Document 6: Wu S, Esumi S, Watanabe K, Chen J, Nakamura K C, Nakamura K, Kometani K, Minato N, Yanagawa Y, Akashi K, Sakimura K, Kaneko T, Tamamaki N. (2011) Tangential migration and proliferation of intermediate progenitors of GABAergic neurons in the mouse telencephalon. Development 138, 2499-2509.
Non-Patent Document 7: Esumi S, Wu S X, Yanagawa Y, Obata K, Sugimoto Y, Tamamaki N. (2008) Method for single-cell microarray analysis and application to gene-expression profiling of GABAergic neuron progenitors. Neurosci Res. 60, 439-451.
Non-Patent Document 8: Tamamaki N, Yanagawa Y, Tomioka R, Miyazaki J, Obata K, Kaneko T. (2003) Green fluorescent protein expression and colocalization with calretinin, parvalbumin, and somatostatin in the GAD67-GFP knock-in mouse. J Comp Neurol. 467, 60-79.
Non-Patent Document 9: Kaartinen V, Dudas M, Nagy A, Sridurongrit S, Lu M M, Epstein J A, (2004) Cardiac outflow tract defects in mice lacking ALK2 in neural crest cells. Development 131, 3481-3490.
Non-Patent Document 10: Ohira K, Furuta T, Hioki H, Nakamura K C, Kuramoto E, Tanaka Y, Funatsu N, Shimizu K, Oishi T, Hayashi M, Miyakawa T, Kaneko T, Nakamura S. Ischemia-induced neurogenesis of neocortical layer 1 progenitor cells. Nat Neurosci. 2010 February; 13(2):173-9. Epub 2009 Dec. 27.
Non-Patent Document 11: Imayoshi I, Sakamoto M, Ohtsuka T, Takao K, Miyakawa T, Yamaguchi M, Mori K, Ikeda T, Itohara S, Kageyama R.(2008) Roles of continuous neurogenesis in the structural and functional integrity of the adult forebrain. Nat Neurosci. 11, 1153-1161.
Non-Patent Document 12: Yu P B, Hong C C, Sachidanandan C, Babitt J L, Deng D Y, Hoyng S A, Lin H Y, Bloch K D, Peterson R T. (2008) Dorsomorphin inhibits BMP signals required for embryogenesis and iron metabolism. Nat Chem Biol. 4, 33-41.

### SUMMARY OF THE INVENTION

### Problems to be Solved by the Invention

The cerebral cortex as a center of human thinking consists of excitatory neurons (80%) and inhibitory neurons (20%). Several mental disorders in human brain are suggested to be caused by loss of the inhibitory neurons (GABAergic neurons). Such the mental disorders are considered to be treated by regenerative medicine in which the inhibitory neurons in an amount sufficient to achieve a therapeutic effect are transplanted to the diseased site in the brain. However, as described above, it is difficult in the current situation to provide the inhibitory neuron progenitors and the developing inhibitory neurons in a sufficient amount. One reason for this is that, while the inhibitory neuron progenitors have a proliferating ability, a means for making the cells proliferate effectively has not been established. Accordingly, the first object of this application is to provide a novel means for making the inhibitory neuron progenitors proliferate effectively (thus, a proliferation promoting factor for the inhibitory neuron progenitors and its use).

When the first object has been accomplished, it becomes possible to obtain the cells in an amount sufficient for a transplantation by culturing and proliferation of the inhibitory neuron progenitors in the brain tissue collected in a small amount from the cerebral cortex of a patient. In such case, however, the cultured material is a cell mixture containing various other neural cells. Otherwise, also when an ES cell or an iPS cell is differentiated into inhibitory neuron progenitors or inhibitory progenitors, the resultant cell population may contain undifferentiated ES cells or iPS cells or neural cells differentiated into other types. Accordingly, from such a cell population, the inhibitory neuron progenitors and the developing inhibitory neurons should surely be separated for the purpose of the transplantation. The second object of this application is to provide a method for separating the inhibitory neuron progenitors and the inhibitory neurons surely from the cell population.

Also once the second object was achieved and it became possible to separate a sufficient amount of inhibitory neuron progenitors or inhibitory neurons for transplantation, it is required to transplant them without significant invasion of the brain and efficiently engraft them. The third object of this application is to provide a method for transplanting the separated inhibitory neuron progenitors or inhibitory neurons without significant invasion of the brain and effectively engrafting them.

The means for achieving the first object is expected to enable a search for a novel factor for making inhibitory neuron progenitors proliferate, whose effect may then further be improved, and also to develop a novel industrially applicable field of the technology. The fourth object of this application is to provide a means for searching for a novel factor for making inhibitory neuron progenitors proliferate.

### Means for Solving the Problems

The inventors acquired the following novel findings as a result of an intensive study to solve the problems set forth above.

(A) Activin A makes inhibitory neuron progenitors proliferate selectively at a high efficiency.

(B) Activin A makes inhibitory neuron progenitors proliferate upon binding to an Activin A receptor, Acvr1 that is expressed only in the inhibitory neuron progenitors in the brain.

(C) When an Activin A-activated inhibitory neuron progenitors is transplanted on the brain surface, it moves into the brain spontaneously.

(D) By pretreatment of the inhibitory neuron progenitors with an Acvr1-specific antibody, the effect of Activin A for making the inhibitory neuron progenitors proliferate is attenuated or lost.

Thus, the present application provides the following Inventions (1) to (6) as means for accomplishing the first object based on the finding (A).

(1) A solution for making inhibitory neuron progenitors proliferate, which contains Activin A.

(2) An in vitro method for making inhibitory neuron progenitors proliferate, which comprises culturing the progenitors in the solution for proliferation of Invention (1).

(3) The in vitro method of Invention (2), wherein the progenitors are cells isolated from a cerebral cortex

(4) The in vitro method of Invention (2), wherein the progenitors are cells differentiated from an ES cell or an iPS cell.

(5) An in vivo method for making inhibitory neuron progenitors proliferate, which comprises bringing an Activin A-containing solution into contact with inhibitory neuron progenitors in brain

(6) The in vivo method of Invention (5), wherein the progenitors are cells distributed adjacent to a brain surface or in a subventricular zone.

Also the present application provides the following Inventions (7) to (9) as means for accomplishing the second object based on the above finding (B).

(7) A method for separating inhibitory neuron progenitors and inhibitory neurons, which comprises isolating Activin A receptor, Acvr1-expressing cells from a population of neurons proliferated under a culture condition.

(8) The method of Invention (7), wherein the population of neurons are isolated from a cerebral cortex and cultured.

(9) The method of Invention (7), wherein the population of neurons are differentiated from ES cells or iPS cells.

Also the present application provides the following Invention (10) as means for accomplishing the third object based on the above finding (C).

(10) A method for transplanting on brain surface the inhibitory neuron progenitors and inhibitory neurons isolated by the method of any one of Inventions (7) to (9).

Also the present application provides the following Invention (11) as means for accomplishing the fourth object based on the above finding (D).

(11) A method for screening a substance that selectively promotes proliferation of inhibitory neuron progenitors, which comprises:
(a) a step for measuring a degree of cell proliferation by contacting a test substance with inhibitory neuron progenitors;
(b) a step for measuring a degree of cell proliferation by contacting the test substance with inhibitory neuron progenitors to which an antibody to an Activin A receptor Acvr1 or a substance inhibiting an intracellular activation of Acvr1 has been preliminarily contacted; and,
(c) a step for selecting the substance as an intended one by which the cell proliferation in step (a) is disrupted or significantly reduced in step (b).

In the respective inventions, the “inhibitory neurons” are neurons releasing an inhibitory neurotransmitter thereby inhibiting the excitation of other neurons. The inhibitory neurons include GABAergic neurons in the cerebrum, diencephalon, mesencephalon and spinal cord, and GABAergic neurons glycinergic neurons in the medulla oblongata. Its “progenitors” are cells having an ability of generating the inhibitory neurons by asymmetric cell division.

The “proliferation” means an increase in the number of cells, as a result of a symmetric cell division, by 2 times, preferably by about 4 times in a state of keeping the fundamental characteristics of the cells. It also means the generation of inhibitory neuron progenitors and inhibitory neurons or inhibitory neuron progenitors and the secondary inhibitory neuron progenitors and the like, as a result of an asymmetric cell division. It also means a manipulation that only the inhibitory neuron progenitors selectively proliferate in a cell population containing inhibitory neuron progenitors isolated from a central nervous system tissue of a mammal including a human or in a cell population containing inhibitory neuron progenitors differentiated from an ES cell or an iPS cell, and a population consisting majorly of inhibitory neuron progenitors and inhibitory neuron is finally obtained.

Other terms and concepts in the present invention will be discussed in the following descriptions of the embodiments as well as Examples.

### Effects of the Invention

According to Invention (1), a solution for making inhibitory neuron progenitors proliferate, which containing Activin A, a novel factor which make inhibitory neuron progenitors proliferate, is provided.

According to Inventions (2) to (4), it is possible, by using the solution of Invention (1), to make the inhibitory neuron progenitors isolated from a cerebral cortex or differentiated from an ES cell or an iPS cell proliferate in vitro, to a large amount.

According to Inventions (5) and (6), by contacting the Activin A-containing solution with the inhibitory neuron progenitors distributed on a brain surface proximity or a subventricular zone, the inhibitory neuron progenitors can be proliferated in the brain (in vivo). This procedure can greatly reduce a burden to a patient compared with a cell transplantation surgery.

According to Inventions (7) to (9), it becomes possible to separate inhibitory neuron progenitors and inhibitory neurons for transplantation exclusively and surely, from a cell population which may contain various types of cells.

According to Invention (10), by transplanting to a brain surface the inhibitory neuron progenitors and the inhibitory neurons separated in Inventions (7) to (9), the inhibitory neuron progenitors and the inhibitory neurons can move by their spontaneous migration ability to a site where they are needed, and hence a burden to a patient is greatly reduced compared with a cell transplantation surgery into a brain parenchyma.

According to Invention (11), it becomes possible to search for a novel substance having a function similar to that of Activin A (thus, a function to bind a receptor Acvr1 and make the inhibitory neuron progenitors proliferate). Such a substance is expected to serve as a safe and effective active ingredient of a brain infusion agent, for example.

### BRIEF DESCRIPTION OF DRAWINGS

FIG. 1 is the results of Experiment 1, a photographic image of a GAD67-GFP mouse cerebral cortex at P0 (a), and PCNA (red) and GFP (green) double labeled photographic image in the SVZ (b). There are many double-labeled cells.

FIG. 2 shows the results of Experiment 2, a pseudo-color indication of the signal intensities from gene probes hit by key words, “Growth factor receptor” in the gene expression profile detected by a single cell microarray analysis method (Non-Patent Document 7).

FIG. 3 shows the results of Experiment 2, a pseudo-color indication of the signal intensities of the collected information only of the Present Call genes judged to be expressed in Cell 2 and Cell 4 among the gene probes hit as Growth factor receptor in the gene expression profile detected by a single cell microarray analysis method (Non-Patent Document 7).

FIG. 4 shows the results of Experiment 2, a measurement of the signal intensity of the Activin A receptors in the gene expression profile detected by a single cell microarray analysis method (Non-Patent Document 7).

FIG. 5 shows the Acvr1 immunoreactive sites detected in a neonatal GAD67-GFP mouse cerebral cortex utilizing a rabbit antibody raised against a peptide from 20 to 50 amino acids chain from the N-terminal of a human Acvr1 (Swiss Prot ID# Q04771). [A] GAD67-GFP positive cells found in the GAD67-GFP mouse P3 subventricular zone and rostral migratory stream. [B] The Acvr1 immunoreactive sites in the same field are labeled in red.

FIG. 6 shows the Acvr1 immunoreactivity observed on the surface of a GFP positive cell as revealed by a confocal microscope.

FIG. 7 shows GABA immunoreactive sites observed in the cerebral cortex of E18 Acvr1^{f+},GAD67-Cre+ mouse fetus and that of E18 Acvr1^{ff},GAD67-Cre+ mouse fetus obtained by mating an Acvr1-flox conditional knock-out mouse with a GAD67-Cre knock-in mouse. While the GABA immunoreactivity in the neocortex of the E18 Acvr1^{f+},GAD67-Cre+ mouse fetus was normal, the GABA stain was reduced greatly in the neocortex of Acvr1^{ff},GAD67-Cre+ in the E18 mouse fetus.

FIG. 8 shows pia-progenitors distributed in the pia mater of a juvenile (P7) GAD67-GFP knock-in mouse. The pia mater was Laminin positive (FIG. 8-C) and appeared in red.

FIG. 9 is the result of Example 1, a photographic image of GFP-BrdU double positive cells which were divided while uptaking BrdU from the culture medium of a neural stem cell culture medium added with Activin A and BrdU. When the GFP positive cells were cultured in the neural stem cell culture medium containing no Activin A, no image like FIG. 9 was obtained.

FIG. 10 shows the results of Example 1, the increase in the DNA amount associated with cell division, which is measured as the BrdU amount incorporated into the DNA when the cells were cultured in the neural stem cell culture medium supplemented with Activin A and BrdU (M+A) or only with BrdU (M). The increase in the DNA amount associated with the cell division was evident especially when Activin A was added to the neural stem cell culture medium.

FIG. 11 shows the infusion of 1 μl of 1 μg/ml Activin A solution to the surface of the cerebral cortex of one side in a 3-day old GAD67-GFP mouse in order to verify the in vivo effect of Activin A. Further after 30 minutes, 20 μl of 10 mM EdU solution was injected intraperitoneally to the mouse. Thereafter, the animal was maintained for 1 week, anesthetized, fixed, and examined histologically. The results indicate that a significantly larger number of the GFP/EdU double-labeled inhibitory neurons (arrow) were observed in the hemisphere infused with Activin A (A-C) compared with the opposite hemisphere (D-F). Also in the pia matter on the infused side, a large number of the cells which were possibly pia progenitors were observed as EdU positive cells.

FIG. 12 shows the enzyme distribution as a color development via a DAB reaction (peroxidase reaction), which is observed by infusing an enzyme, microperoxidase, into a large subarachnoid cavity called a large cistern on the caudal side of the mouse cerebellum and by fixing with formalin 30 minutes later. In the case of the microperoxidase being not able to penetrate, a barrier corresponding to the blood brain barrier is judged to be present.

FIG. 13 shows diagrammatically a method for separating inhibitory neuron progenitors and inhibitory neurons from other cells. Method: [1] A cerebral cortex is excised from a neonatal GAD67-GFP mouse and treated with trypsin to gently degrade the cell surface protein molecules thereby distributing the cells to yield a GAD67-GFP positive cell (inhibitory neuron progenitors and inhibitory neurons)-containing cell suspension. [2] A rabbit antibody to the Acvr1 N-terminal amino acids chain was added to the cell suspension. [3] A goat antibody conjugated with biotin molecules that is capable of binding to the rabbit antibodies is added. [4] A turbid fluid of streptavidin-magnetic beads capable of binding to the biotin is added. [5] As a result, the magnetic bead was bound to the Acvr1 N-terminal amino acids chain via the rabbit antibody, the goat antibody, the biotin molecule and Streptavidin. [6] The fluid-containing tube is brought close to a magnet to separate the Acvr1 positive cells.

FIG. 14 shows that the Acvr1 positive cells can be separated by the method in FIG. 13. The Hoechst blue fluorescence allows the intranuclear DNA to be visible and reveals all cells. The GFP green fluorescence indicates selected inhibitory neuron progenitors and inhibitory neurons. In the field of this photograph, the both cells were in a complete agreement.

FIG. 15 shows that a CAG-ImRFPIGFP-polyA plasmid DNA was introduced into basal ganglion primordium of a GAD67-Cre mouse fetus E14 by an electroporation, and the GAD67 positive cells migrating from the basal ganglion primordium to the cerebral cortex were labeled. A part of the migrating cells were inhibitory neuron progenitors with proliferation activity (Non-Patent Document 6) which is divided on the outer surface of the pia matter (FIG. 15-A) to become the pia-progenitors, and also after growth they are maintained on the brain surface and in the pia matter while various gene expressions are ceased (FIG. 15-B, C). MZ: Marginal zone, Pia: Pia matter, Borderline: Basal lamina.

FIG. 16 shows cells in a Nestin-CreER/GFP adult mouse brain, and visualized by administering Tamoxifen, inserting an electrode into the amygdala on the one side to stimulate the brain, and thereby forming a kindling. At every electric stimulation, BrdU was injected intraperitoneally. As the result of these procedures, inhibitory neuron progenitors became nestin positive and gave rise inhibitory neurons, which were stained with GFP and BrdU. An arrow in the figure indicates that the pia progenitors on the pia matter surface uptook BrdU thereby becoming Nestin positive and then the cells divided and moved from the brain surface to the marginal zone while differentiating into inhibitory neurons.

FIG. 17 is a schematic view indicating that while the cell proliferation was promoted by the binding of Activin A to the Activin A receptor Acvr1, the cell proliferation was not promoted when the antibody was bound to the Activin A binding site of Acvr1 to prevent Activin A binding. (A) The GFP-positive cells contained in the cerebral cortex of a neonatal GAD67-GFP mouse were separated by using a cell sorter, which were then cultured for 2 days in the culture medium [M] for neural stem cells, and the EdU uptaken into the DNA of the cells was visualized by a color development on a blotting membrane. (B) The same GFP-positive cells as cells used in (A) were cultured for 2 days in the medium for the neural stem cells plus overnight-conditioned culture medium [M+C] containing secreted various proliferation factors. And the EdU uptaken into the DNA of the cells was visualized by a color development on a blotting membrane. (C) Under the condition in (B) but containing this time an anti-Activin A blocking antibody additionally, the cells were cultured for 2 days and the EdU uptaken into the DNA of the cells was visualized by a color development on a blotting membrane. (D) Under the condition in (B) but containing this time a rabbit antibody against the peptide of 20 to 50 amino acids from the N terminal of a human Acvr1 (Swiss Prot ID# Q04771) additionally, the cells were cultured for 2 days and the EdU uptaken into the DNA of the cells was visualized by a color development on a blotting membrane.

### MODE FOR CARRYING OUT THE INVENTION

The proliferating solution for inhibitory neuron progenitors of Invention (1) can be prepared by adding Activin A to a culture solution for a neural cell. The Activin A content in the culture solution for a neural cell is within the range of 1 μg to 100 μg/ml, preferably 10 μg to 40 μg/ml, more preferably 15 μg to 25 μg.

Activin A can be obtained by the methods described for example in Patent Documents 1 to 3.

The culture solution for neural cells may be any known culture medium employed for culturing various nervous system cells, such as, for example, a salt solution containing a basic fibroblast growth factor (bFGF) at 20 μg/ml and an epidermal growth factor (EGF) at 20 μg/ml (Neurobasal Medium). Also for the purpose of the present invention (proliferation of the inhibitory neuron progenitors), it is preferred for the medium to add an insulin, an insulin-like growth factor 1, an insulin-like growth factor 2, a leukemia inhibitory factor (LIF), androgen and estrogen, based on the “single cell microarray data” described below.

The in vitro proliferation methods of Inventions (2) to (4) are characterized by culturing the inhibitory neuron progenitors in the solution of Invention (1), and making the cells proliferate.

The inhibitory neuron progenitors to be cultured are those of differentiated from cells in a brain tissue such as a cerebral cortex (proximity to the brain surface, subventricular zone and the like) or an ES cell or an iPS cell by known methods (for example, Non-Patent Documents 1 and 2).

The cell culture can be performed similarly to the known procedures for culturing neurons. According to the method of the present invention, the inhibitory neuron progenitors can be increased by 2 to 4 times by an about 1-week culture.

The in vivo proliferation methods of Inventions (5) and (6) are characterized by contacting the Activin A-containing solution with the inhibitory neuron progenitors in brain. The solution itself may be identical to that of Invention (1) and the Activin A concentration is preferably equal to or higher by several times than that of the solution of Invention (1). For infusion into the brain surface, a high molecular weight polymer having a reduced biological effect such as a human collagen may be mixed with the solution to ensure reducing flowability and localizing. In the case of the solution being directly infused into the cerebral cortex, a prolonged period for the contact with and the penetration into the brain should be ensured. For infusion into a ventricle, the solution can be administered as it is. This method typically allows the inhibitory neuron progenitors distributed to the proximity to the brain surface or the subventricular zone to proliferate as a result of the infusion of the Activin A-containing solution into the cranial cavity or the ventricle via a catheter and the like. The Activin A infusion volume is about 50 ml to 500 ml, which is infused over a period of 30 minutes to 5 hours.

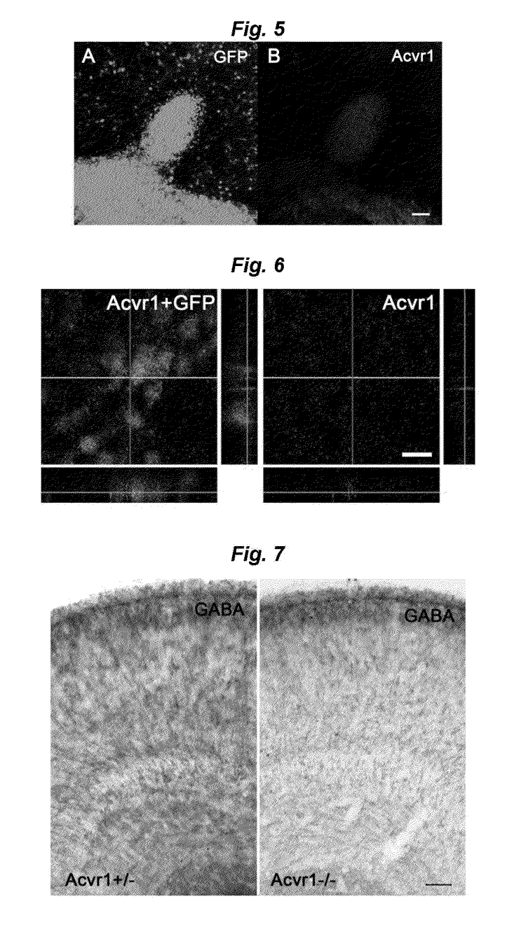

Inventions (7) to (9) are methods for separating the inhibitory neuron progenitors and the inhibitory neurons by isolating cells which are expressing Activin A receptor Acvr1 from a cell population proliferating under a culture condition. The neuron population is a cell population isolated from a cerebral cortex and then cultured, or a cell population differentiated from an ES cell or an iPS cell. Thus, while several genes are known as Activin A receptors, Acvr1 is a receptor expressed only by the inhibitory neuron progenitors and inhibitory neurons in the cerebral cortex (FIGS. 5 and 6), and hence it is possible by selecting the cells expressing the Acvr1 to separate the inhibitory neuron progenitors and the inhibitory neurons from other cells. Typically, for example, the inhibitory neuron progenitors and the inhibitory neurons specifically expressing Acvr1 can be separated by binding a magnetic bead to target cells via an antibody to Acvr1 and then recovering the magnetic bead using a magnet, thereby separating the inhibitory neuron progenitors and the inhibitory neurons.

Alternatively, an anti-Acvr1 antibody is labeled for example with a fluorescent dye and then a fluorescence-activated cell sorter (FACS) is used to separate the inhibitory neuron progenitors and the inhibitory neurons specifically expressing the Acvr1.

Invention (10) is a method for transplanting the inhibitory neuron progenitors and the inhibitory neurons separated by the methods of Inventions (7) to (9) to the brain surface. The separated inhibitory neuron progenitors and the inhibitory neurons before forming an axon or a dendrite have migration ability. The inhibitory neuron progenitors and the inhibitory neurons are mixed with a high molecular weight polymer such as a human collagen having a reduced biological effect to prepare a cell mixture solution of which flowability is reduced and local residence are ensured, and then infused into the brain surface or a cerebral cortical sulcus. During the contact of the cell mixture solution with the brain surface, the inhibitory neuron progenitors and the inhibitory neurons having the migration ability penetrate the pia matter to enter the cerebral cortex parenchyma, and then move to inhibitory neuron-defective region where they are engrafted to be incorporated into the nervous circuit.

A method of invention (11) is a method screening a substance that selectively promotes proliferation of inhibitory neuron progenitors (that is, an Activin A-receptor Acvr1 binding-mediated proliferation promoting substance), comprising the following steps:
(a) a step for measuring a degree of cell proliferation by contacting a test substance with inhibitory neuron progenitors;
(b) a step for measuring a degree of cell proliferation by contacting the test substance with inhibitory neuron progenitors to which an antibody to an Activin A receptor Acvr1 or a substance inhibiting an intracellular activation of Acvr1 has been preliminarily contacted; and,
(c) a step for selecting the substance as an intended one by which the cell proliferation in step (a) is disrupted or significantly reduced in step (b). Thus, several substance for promoting the proliferation of inhibitory neuron progenitors are known, and serve for proliferation of several types of progenitor cells including the inhibitory neuron progenitors. However, for the purpose of proliferating only the inhibitory neuron progenitors exclusively in a cell population, a substance which exerts the proliferation activity as a result of binding to the receptor Acvr1, such as Activin A, is required. Even if a substance inducing proliferation of the inhibitory neuron progenitors is obtained in the step (a), it is required to verify that this substance is an Activin A-receptor Acvr1 binding-mediated one. Accordingly, by inhibiting the binding of the test substance to Acvr1 in step (b) and measuring the effect of the test substance on proliferation, it is finally possible in step (c) to confirm that the test substance is an Activin A-Acvr1 binding-mediated proliferation promoting substance.

In step (a), a proliferation degree of the inhibitory neuron progenitors may be verified, as shown in the following Examples, by measuring an uptake of the thymidine triphosphate analogue (BrdU or EdU) as an index. The test substance may for example be an organic or inorganic compound (especially a low molecular weight compound), a protein, a peptide and the like. These substances may have known or unknown functions or structures. Alternatively, a “combinatorial chemical library” is a useful tool as a candidate substance group for identifying an intended substance efficiently. The combinatorial chemical library is a collection of various chemical composition produced by binding a large number of chemical “building blocks” such as reagents by means of a chemical synthesis or a biological synthesis. For example, a linear combinatorial chemical library such as a peptide library is generated by binding the building block (amino acid) sets in possible all manners for a predetermined length of the compound (thus the size of the peptide). Thorough such a combinatorial mixing of the chemical building blocks, a large number of the chemical compositions can be synthesized. For example, a systematic combinatorial mixing of 100 exchangeable chemical building blocks results in a hundred million tetramer compounds or a ten thousand million pentamer compounds (for example, see Gallop et al., (1994) 37(9):1233-1250). The preparation and screening of the combinatorial chemical library is known in the art (for example, see U.S. Pat. No. 6,004,617 and 5,985,365). Also, various kinds of available libraries may be employed.

In step (b), the purpose is to inhibit the Acvr1 from binding to the test substance specifically, and to specifically inhibit the change in the Acvr1 intracellular region generated as a result of the specific binding of the Acvr1 to the test substance. The former inhibition is, as shown in Example 5, capable of inhibiting the Acvr1 from binding to the test substance by adding a rabbit antibody against a peptide of 20 to 50 amino acids from the N terminal of a human Acvr1 (Swiss Prot ID# Q04771). While the antibody may be a polyclonal antibody, a monoclonal antibody is employed preferably. The latter inhibition is to verify whether the test substance is an Activin A-Acvr1 binding-mediated specific proliferation promoting substance by means of a pretreatment of the inhibitory neuron progenitors with a substance (for example, 6-[4-(2-piperidin-1-ylethoxy)phenyI]-3-pyridin-4-ylpyrazolo[1,5-a]pyrimidine) which enter the gap of the protein structure of the Acvr1 intracellular region to interfere with the change therein thereby inhibiting the signal transmission even if the Acvr1 is bound to the test substance (Non-Patent Document 12).

In step (c), the larger in the uptake amounts of the thymidine triphosphate analogue (BrdU or EdU) into the generated inhibitory neuron progenitors is measured, the more hopeful and specific substance for promoting proliferation of the inhibitory neuron progenitors can be specified.

Hereinafter, the experiments conducted for investigating the relationship of the inhibitory neuron progenitors (GABAergic neuron progenitors) with Activin A and a receptor thereof are shown together with Examples to describe the invention of the present application typically in more detail, but the invention of the present application is not limited to the followings.

### EXPERIMENT 1

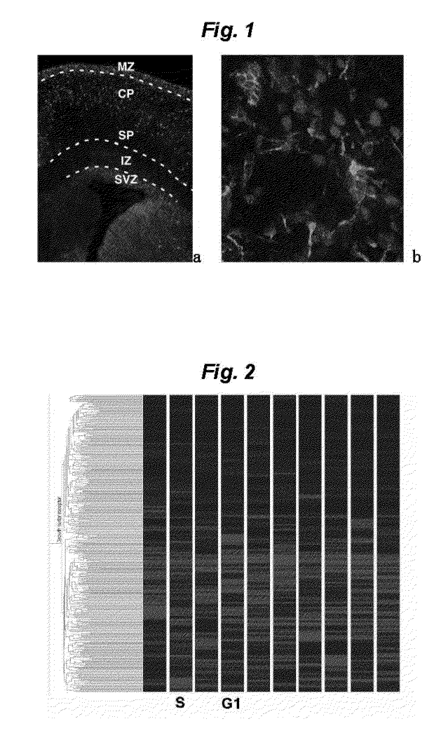

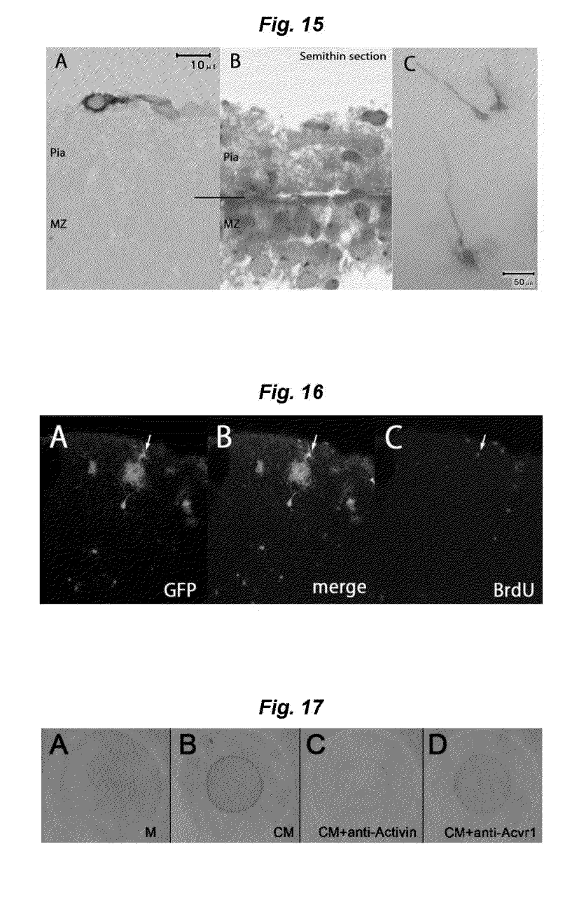

In order to investigate the nature of inhibitory neuron progenitors, GFP positive cells in the cerebral cortex immediately after the birth of a GAD67-GFP knock-in mouse to which the GFP was inserted into the GAD67 (GABA synthetase) locus (Non-Patent Document 8) (FIG. 1a). As a result, it was revealed that the GFP-positive cells in the mice express both neuron markers (for example GAD67 and doublecortin) and cell cycle markers (PCNA, Ki-67, cyclinD1/2, cyclinE1/2, phospholirated-HistonH3), and uptake BrdU during the DNA synthetic phase (FIG. 1b). Such the cells in the above were judged to be the inhibitory neuron progenitors, whose distribution was then investigated. As a result, it was found that a large number of the inhibitory neuron progenitor were present in the subventricular zone, the intermediate zone (Non-Patent Document 6) as well as, based on this experiment, in the cortical plate, the marginal zone and the pia mater (FIGS. 15 and 16) of the P0 mouse cerebral cortex.

### EXPERIMENT 2

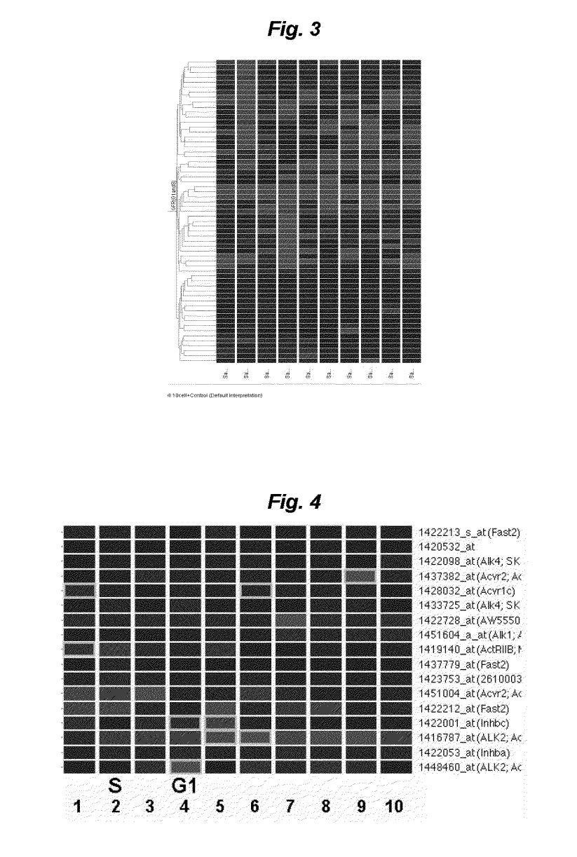

Then, a part of the SVZ of the cerebral cortex in the GAD67-GFP P0 mouse was taken out, treated with a protease (trypsin) to disperse the cells, from which 10 GFP-positive cells were picked up and subjected to a single-cell microarray analysis (Non-Patent Document 7). As a result, the Nos. 1 to 5 cells were in a state of cell cycle, especially No. 2 cells were in the S phase of the cell cycle in view of the situation of the expression of cyclin E1/E2, PCNA, many DNA synthesis-related genes, and No. 4 were in G1 phase in view of the cyclin D1/D2 expression (Non-Patent Document 7). Accordingly, based on the keyword of “the cell proliferation factor receptor” expressed by the 10 cells, 650 candidate genes were selected and shown collectively (FIG. 2). As a result, it was revealed that the expression levels of the cell proliferation factor receptor are different according to each phase in the cell cycles of the respective cells. Cells 1 to 5 are Notch-3 negative and suspected to be ready to undergo a symmetric cell division, and Cells 6 to 10 are Notch-3 positive and suspected to be ready to undergo an asymmetric cell division or to have undergone the asymmetric cell division. Therefore, Cells 6 to 10 may possibly be the inhibitory neurons. Even when a cell proliferation factor receptor is expressed in a neuron, it may be associated with the extension of dendrites or the maturity of the cells instead of being associated with the cell division. On the contrary, G1 phase is in an important check point period after which the cell division is terminated and the differentiation is initiated, or the division phase is re-entered. Therefore, the factor which binds to the cell proliferation factor receptor which was judged to be expressed in Cell 4 in G1 phase (Present Call) may play an important role for regulating the cell division. Therefore, the list of the cell proliferation factor receptor genes judged to be expressed in Cell 4 was combined with the list of the cell proliferation factor receptor genes judged to be expressed in Cell 2 which was in S phase during a DNA replicating phase to prepare the figure (FIG. 3).

When inspecting FIG. 3, it was revealed that the Cell 4 expressed Activin A receptor type 1 (Acvr1:ALK2) as a characteristic molecule. Furthermore, in addition to the Activin A receptor Acvr1, the receptors for basic fibroblast growth factor (bFGF), epidermal growth factor (EGF), Insulin, Insulin-like growth factor 1 (AF440694), Insulin-like growth factor 2 (AK011784), Leukemia inhibitory factor (LIF; AF065917), Androgen receptor, Estrogen receptor were detected, and the activities of these receptors are considered to be required for maintaining the cell proliferation. The Activin A receptor which was focused on as a characteristic molecule is on the list in FIG. 4.

### EXPERIMENT 3

It was verified whether the Activin A receptor Acvr1 was expressed actually in the inhibitory neuron progenitors. An antibody prepared by immunizing a rabbit using as an antigen a peptide of 20 to 50 amino acids from the N terminal of a human Acvr1 (Swiss Prot ID# Q04771) was employed. This antibody was used to detect Acvr1 on the mouse cerebral cortex. As a result, the Acvr1 immunoreactivity was found in the subventricular zone of the cerebral cortex where a large number of juvenile inhibitory neurons and inhibitory neuron progenitors were distributed (FIG. 5).

A 3D-presentation at a higher magnification revealed that the stain was localized in a part of the GFP positive cell surface (FIG. 6).

Then, the Acvr1 was deleted only in the GAD67 positive cells. A knock-out mouse with a systemic Acvr1 deletion cannot be employed for an appropriate experiment because of its fetal lethality. Therefore, in this experiment, a mouse having a P1 phage-derived DNA, loxP, inserted into the both sides of the 7th exon of Acvr1 gene (conditional knock-out mouse) (Non-Patent Document 9) was obtained from Michigan University, U.S.A., and a GAD67-Cre knock-in mouse (Non-Patent Document 6) was obtained from Niigata University, and the both mice were mated to make the Acvr1 expression to be deleted only in the GAD67 positive cells. As a result, a postnatal death was observed because of no clear reason. Therefore, on the day before the birth, the fetus whose Acvr1 expression had been deleted only in the GAD67 positive cells on the 18th embryonic day (E18) was taken out and fixed with perfusion, and the immunohistological examination for GABA in the cerebral cortex was conducted. The results showed a substantially reduced GABA immunohistological stain as in FIG. 7.

### EXPERIMENT 4

The presence of the inhibitory neuron progenitors in the subventricular zone around the ventricle in brain was indicated in FIG. 1. In order to investigate the distribution of the inhibitory neuron progenitors to the proximity to the brain surface, the pia matter of a juvenile (P7) GAD67-GFP knock-in mouse was examined. In the pia matter, a large number of the GFP positive cells were distributed, which are referred to as pia-progenitors. In FIG. 8, the pia matter is laminin positive (FIG. 14-C), and appears red.

### EXAMPLE 1

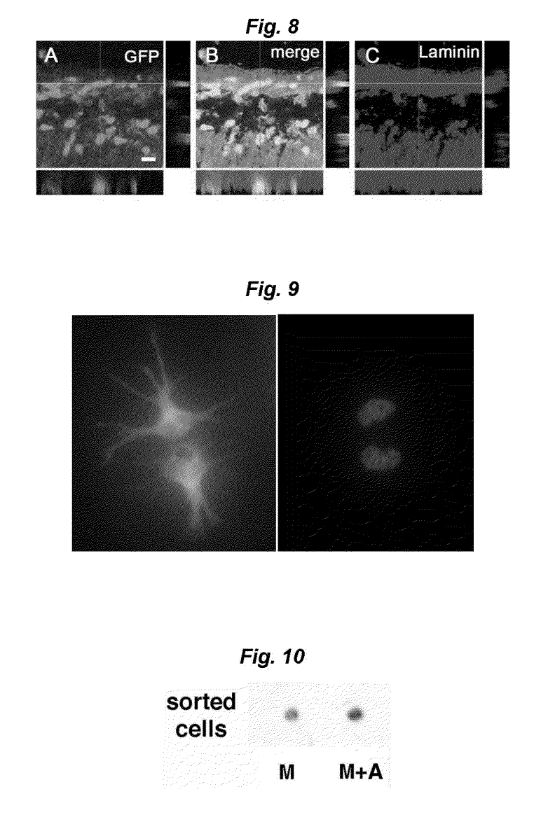

The cerebral cortex of a GAD67-GFP P0 mouse was taken out, treated with a protease (trypsin) to disperse the cells, which were subjected to a cell sorter to obtain GFP positive cells. A neural stem cell culture medium (Neurobasal medium +5% B27 supplement, 20 ng/ml bFGF, 20 ng/ml EGF) was combined with a E18 mouse fetal brain tissue and cultured overnight for conditioning, thereafter the cells were removed and then 5 μg/ml of BrdU was added and the GFP positive cells taken out by the cell sorter were cultured for 2 to 7 days and then fixed. As a result, a part of the GFP positive GABAergic neuron progenitors was confirmed to uptake BrdU in a conditioned neural stem cell culture medium to undergo a cell division (FIG. 9). When the GFP positive cells were cultured in a neural stem cell culture medium which had not been conditioned with the fetal brain tissue, the image as of FIG. 9 was not obtained.

Based on the above results, it was suggested that while the GABA neuron progenitors cannot proliferate in the neural stem cell culture medium. The proliferation may be successful in the neural stem cell culture medium supplemented with essential proliferation factors, thus establishing an essential proliferation factor detection system. The uptake of BrdU into the DNA in the neural stem cell culture medium with or without Activin A at 20 ng/ml was measured, and the results indicated a significant difference (FIG. 10). Based on these results, it was proven that binding of an agonist to the Activin A receptor Acvr1 (1448460-ALK2) expressed in G1 phase promotes the proliferation of the inhibitory neuron progenitors. While 1416787-ALK exhibited a high signal in most of the 10 cells and also gave Present Call in Cell 5 and Cell 6, it may be another Acvr1 splicing variant since the probe was different.

### EXAMPLE 2

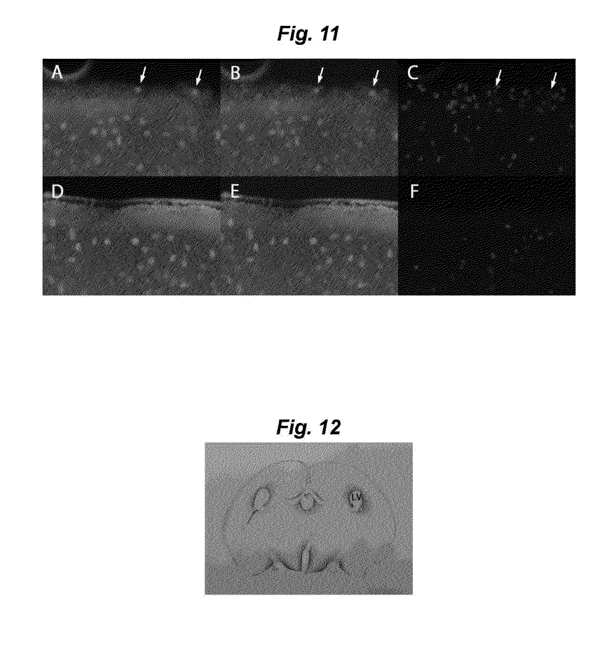

Also in order to verify the in vivo effect of Activin A, an Activin A solution was injected directly into the brain of a 3-day old (P3) juvenile mouse. Thirty minutes after this addition, EdU was injected intraperitoneally. Thereafter, the animal was maintained for 1 week and then fixed under anesthesia for the histological observation, which revealed a significantly larger number of the GFP/EdU double-stained inhibitory neurons (arrow) in the hemisphere into which Activin A was infused (A to C), compared with the opposite hemisphere (D to F). In the pia matter on the infusion side, a large number of the cells considered to be the pia progenitors (considered to be GFP/EdU positive initially) were observed as the EdU positive cells (FIG. 11). These results suggested that if it is possible to contact an Activin A-containing solution with the inhibitory neuron progenitors in the brain then the inhibitory neuron progenitors can proliferate in vivo. Then the subsequent issue is a method for delivering Activin A to the inhibitory neuron progenitors inside the brain. Such a method is preferably a method avoiding any damage to the brain, such as penetration of an injection needle.

It has already discussed that the most of the inhibitory neuron progenitors were distributed to the proximity to the brain surface or the subventricular zone. In order to establish a technique for delivering Activin A to the proximity to the brain surface or the subventricular zone, a protein which can readily be detected was employed instead of Activin A itself in a monitoring experiment. The protein employed was a microperoxidase which was a peptide obtained by enzymatic degradation of cytochrome C and purification of a heme-containing moiety. A caudal part of a mouse cerebellum has a large subarachnoid cavity referred to as the cistern magna, into which the microperoxidase was infused, and after 30 minutes the formalin fixing was conducted and the enzyme distribution was visualized by a color development via DAB reaction (peroxidase reaction). It is defined that when the microperoxidase cannot permeate then a barrier corresponding to the blood brain barrier exists. The experimental results shown in FIG. 12 suggest that a part of the microperoxidase entered from the cistern to the fourth ventricle, and then from the fourth ventricle to the third ventricle, and then reached the lateral ventricle (LV), and then migrated from the surface of the lateral ventricle to the brain parenchyma. It is also indicated that the microperoxidase going from the cistern circumferentially to the basilar part also permeated from the pia matter into the brain parenchyma. It means that there was no barrier corresponding to BBB between the subarachnoid cavity and the brain parenchyma (the boundary is the pia matter) or between the ventricle and the brain parenchyma (the boundary is a ventricular zone). The same results were obtained when using a horse radish peroxidase having a larger molecular weight instead of the microperoxidase. Thus, by the method for infusing a solution containing Activin A or other cell proliferation factors into the brain surface (subarachnoid cavity) or the ventricle, Activin A or other cell proliferation factors can be contacted with and can exert their effects on the inhibitory neuron progenitors distributed in the proximity to the brain surface and the subventricular zone.

### EXAMPLE 3

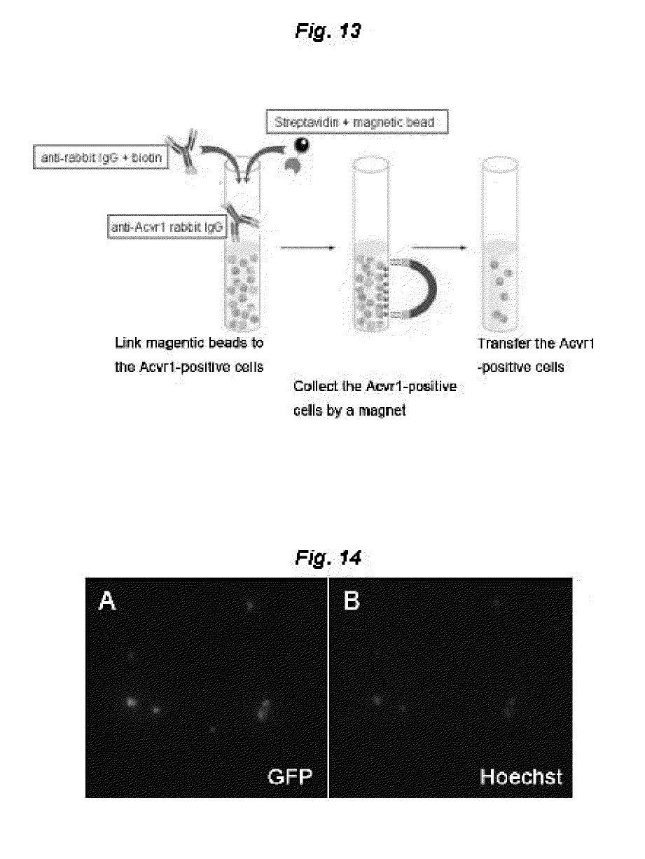

Acvr1 is a membrane protein, has an extracellular Activin A binding site which penetrates the cell membrane, and is a molecule having an intracellular function for phosphorylating other molecule. Accordingly, the anti-Acvr1 antibody employed in Experiment 3 can bind to Acvr1 while allowing the cell to be still alive. Since Acvr1 is expressed only in the inhibitory neuron progenitors and the inhibitory neurons in the brain (FIGS. 5 and 6), the method shown schematically in FIG. 13 was employed to separate the inhibitory neuron progenitors and the inhibitory neurons.

The results are shown in FIG. 14, which indicates that the GAD67-GFP positive cells (the inhibitory neuron progenitors and the inhibitory neurons) were separated successfully as major cells.

### EXAMPLE 4

The inhibitory neuron progenitors and the inhibitory neurons were transplanted as being in contact with the brain surface. Instead of describing all steps of the transplantation, the steps which had not been verified were described exclusively, while referring to the already verified events just by designating the respective supportive articles.

Into the cerebral ganglion primordium of a GAD67-Cre fetal mouse E14, a CAG-ImRFPIGFP-polyA plasmid DNA was electroporated, and the cells belonging to the GAD67 positive cell lineage which migrated from the cerebral ganglion primordium to a cerebral cortex were labeled with GFP. A part of the migrating cells were proliferative competent inhibitory neuron progenitors (Non-Patent Documents 6), in which those of coming to the brain surface then penetrated through the basal lamina layer into the pia matter (FIG. 15a-b), and became pia-progenitors which then underwent repetitive cell division (FIG. 15c). Thus, the inhibitory neuron progenitors were revealed to have an ability of penetrating the basal lamina layer. The basal lamina layer has not been reported to have a polarity, and the inhibitory neuron progenitors can protrude from the brain and also can protrude into the brain.

While it is preferable that the inhibitory neuron progenitors and the inhibitory neurons transplanted to the brain surface are delivered further deeply into the cerebral cortex, it was reported that the brain has a system to allow the endogenous progenitors present near the brain surface to proliferate and to be delivered to the deeper layers in the cerebral cortex (Non-Patent Document 10).

In this Example, a Nestin-CreER mouse was contributed from Kyoto University and used for an Experiment, which mouse had been employed in a study on the production of olfactory bulb granular cells (inhibitory neuron) in a cerebral cortex subventricular zone (Non-Patent Document 11). This mouse and a GFP Cre-reporter mouse were mated to establish a mouse having the both genes. To this mouse, Tamoxifen was administered and an electrode was unilaterally inserted into the amygdala to stimulate the brain thereby forming kindling to yield an epileptic mouse.

At every electric stimulation, BrdU was injected intraperitoneally and the inhibitory neurons and the inhibitory neuron progenitors were stained with GFP and BrdU, in which the inhibitory neuron progenitors had become nestin-positive and produced the inhibitory neuron progenitors. On the outer surface of the brain, the neuron progenitors were BrdU positive as shown in FIG. 16, while the later observation of other mice revealed that BrdU+GFP double-positive cells appeared on the marginal layer. Also thereafter, the GFP positive cells were distributed further deeply, confirming the details reported in Non-Patent Document 10 again.

The results described above assure, based on the known fact in Non-Patent Documents 10 and our finding that the inhibitory neuron progenitors and the inhibitory neurons can penetrate the basal lamina, that a safe transplantation of the inhibitory neuron progenitors and the inhibitory neurons onto the brain surface is feasible.

### EXAMPLE 5

While the cell proliferation was promoted by the binding of Activin A to the Activin A receptor Acvr1, the cell proliferation was not promoted when an antibody was bound to the Activin A binding site of Acvr1 to prevent Activin A binding (FIG. 17).

(A) First, the GFP positive cells (the inhibitory neuron progenitors and the inhibitory neurons) contained in the cerebral cortex of a neonatal GAD67-GFP mouse were separated by cell sorter, then cultured for 2 days in the neural stem cell-directed culture medium (M), and the EdU uptake into the DNA of the cells was subjected to a color development on a blotting membrane, but the EdU uptake observed was only at about the detection limit (FIG. 17-A). This state corresponds to the situation where the binding of Activin A to Acvr1 is inhibited completely.

(B) Then, a neonatal mouse cerebral cortex tissue was cultured overnight in the neural stem cell-directed culture medium, which was allowed to secrete various proliferation factors to provide a conditioned culture medium (M+C), in which the inhibitory neuron progenitors were cultured, and as described in (A), the EdU uptake into the DNA was visualized by a color development on a blotting membrane (FIG. 17-B).

(C) Then, under the condition (B) but containing an anti-Activin A blocking antibody additionally, the cells were cultured for 2 days and the EdU uptake into the DNA of the cells was subjected to a color development on a blotting membrane for quantification (FIG. 17-C).

(D) Finally, under the condition (B) but containing an rabbit antibody against the peptide of 20 to 50 amino acids from the N terminal of a human Acvr1 (Swiss Prot ID# Q04771) additionally, the cells were cultured for 2 days and the EdU uptake into the DNA of the cells were visualized by a color development on a blotting membrane (FIG. 17-D).

Based on the comparison of the above results, the conditioned culture medium (M+C) contains Activin A and becomes to be in a condition similar to that of the neural stem cell-directed culture medium by depleting Activin A using the antibody.

It was shown that when adding the rabbit antibody against the peptide of 20 to 50 amino acids from the N terminal of human Acvr1 (Swiss Prot ID# Q04771), Activin A was not able to bind to Acvr1 and the inhibitory neuron progenitors became impossible to proliferate even in the presence of Activin A. Activin A is a ligand for Acvr1 and any ligand as an alternative to Activin A can be identified by the method similar to that described above.

1. A solution for making inhibitory neuron progenitors proliferate, which contains Activin A.

2. An in vitro method for making inhibitory neuron progenitors proliferate, which comprises culturing the progenitors in the solution for proliferation of claim 1.

3. The in vitro method of claim 2, wherein the progenitors are cells isolated from a cerebral cortex.

4. The in vitro method of claim 2, wherein the progenitors are cells differentiated from an ES cell or an iPS cell.

5. An in vivo method for making inhibitory neuron progenitors proliferate, which comprises bringing an Activin A-containing solution into contact with inhibitory neuron progenitors in brain.

6. The in vivo method of claim 5, wherein the progenitors are cells distributed adjacent to a brain surface or in a subventricular zone.

7. A method for separating inhibitory neuron progenitors and inhibitory neurons, which comprises isolating Activin A receptor, Acvr1-expressing cells from a population of neurons proliferated under a culture condition.

8. The method of claim 7, wherein the population of neurons are isolated from a cerebral cortex and cultured.

9. The method of claim 7, wherein the population of neurons are differentiated from an ES cell or an iPS cell.

10. A method for transplanting on brain surface the inhibitory neuron progenitors and inhibitory neurons isolated by the method of claim 7.

11. A method for screening a substance that selectively promotes proliferation of inhibitory neuron progenitors, which comprises:
(a) a step for measuring a degree of cell proliferation by contacting a test substance with inhibitory neuron progenitors;
(b) a step for measuring a degree of cell proliferation by contacting the test substance with inhibitory neuron progenitors to which an antibody to an Activin A receptor Acvr1 or a substance inhibiting an intracellular activation of Acvr1 has been preliminarily contacted; and,
(c) a step for selecting the substance as an intended one by which the cell proliferation in step (a) is disrupted or significantly reduced in step (b).

12. A method for transplanting on brain surface the inhibitory neuron progenitors and inhibitory neurons isolated by the method of claim 8.

13. A method for transplanting on brain surface the inhibitory neuron progenitors and inhibitory neurons isolated by the method of claim 9.
